(19) United States
(12) Patent Application Publication
(10) Pub. No.: US 2021/0023090 A1
(43) Pub. Date: Jan. 28, 2021

(54) Antipruritic Pharmaceutical Promethazine Hydrochloride Compositions for Topical Spray-On Application

(71) Applicant: Kelly Jean Cooper, Cedarburg, WI (US)

(72) Inventor: Kelly Jean Cooper, Cedarburg, WI (US)

(21) Appl. No.: 16/811,561

(22) Filed: Mar. 6, 2020

Related U.S. Application Data
(60) Provisional application No. 62/818,675, filed on Mar. 14, 2019.

(51) Int. Cl. A61K 31/5415; A61K 47/46; A61K 9/00

(52) U.S. Cl. CPC A61K 31/5415; A61K 9/0014; A61K 47/46

(57) ABSTRACT

A topical pharmaceutical preparation suitable for use as an antipruritic drug comprises an aloe vera liquid and a therapeutic amount of promethazine hydrochloride. The preparation is preferably packaged in an airless spray container. A method for preparing the topical pharmaceutical preparation comprises the step of adding a therapeutic amount of promethazine hydrochloride to aloe vera liquid. The preparation is suitable to stop itching due to skin irritations without the need for reapplication and requires few ingredients and few steps to manufacture.

### CROSS-REFERENCE TO RELATED APPLICATIONS

This application claims the benefit of provisional patent application Ser. No. 62/818,675, filed 2019 Mar. 14 by the present inventor, which is incorporated by reference in its entirety.

### TECHNICAL FIELD

The present invention relates to pharmaceuticals, and more particularly to a topical pharmaceutical preparation suitable for use as an antipruritic drug.

### BACKGROUND-DISCUSSION OF PRIOR ART

I have found that there is a need in the pharmaceutical industry to create a preparation of an antipruritic drug for topical application that is long-lasting without the need for reapplication, is not displeasing to the touch, uses a small amount of packaging, and is inexpensive to manufacture.

Currently there are topical products that relieve itching due to insect bites, hives, and other mild skin irritants. These products contain ingredients such as baking soda and alcohol to counteract the itching. These ingredients only provide temporary relief and require frequent reapplication.

Antihistamines are very effective at reducing itching. The few existing topical products use diphenhydramine as the antihistamine. These products only provide temporary relief and require reapplication, similar to products containing baking soda and alcohol. Consumers also complain of how these products feel on their skin because it is sticky to the touch.

Promethazine hydrochloride is a longer-lasting antihistamine than diphenhydramine. Previous attempts at using promethazine hydrochloride topically have not adequately addressed the problem of making an application that does not have to be rubbed into the skin, thus getting the consumer's hands full of product. Additionally, these products require many ingredients to make the full product. For example, published U.S. Pat. App. No. 2008/0255103 A1. to Aslum, Nulu, Ballay, and Ballay teaches a topical antihistamine gel in a single unit dose packaging with one embodiment comprised of purified water, potassium sorbate, methyl paraben sodium, sodium citrate, disodium EDTA, promethazine hydrochloride, and alpha-lipoic acid. I have found that not only is it expensive to make a product with this many different ingredients, it also takes several steps to manufacture and thus is time consuming. Another disadvantage is that since this product comes in single unit dose packaging, it requires quite a lot of material to produce several applications. This is not feasible for pruritus due to causes such as mosquito bites. A person with several bites would have to use several packages. This is an added cost to manufacturing and makes the product less convenient for consumers to use and take with them.

### SUMMARY

In accordance with one embodiment of the present invention, a topical pharmaceutical preparation suitable for use as an antipruritic comprises an aloe vera liquid base and a therapeutic amount of promethazine hydrochloride. The aloe vera liquid is a natural antipruritic that provides immediate relief of itching while the promethazine hydrochloride takes effect to stop the itching long term by stopping the body from producing histamine, the chemical in the body that causes itching.

In accordance with a further embodiment of the present invention, the topical pharmaceutical preparation of the present invention is packaged in an airless spray container. The preparation may be packaged in an airless pump bottle or in a bag-on-valve system.

### Advantages

Accordingly, several advantages of one or more aspects are as follows: to provide a topical antipruritic that is long-lasting, that stops the itching after the first application and does not need additional applications, that does not leave the hands feeling sticky, that does not need many packaging materials, and that does not need many ingredients.

### Drawings-Figures

The foregoing summary, as well as the following detailed description of preferred embodiments of the invention, will be better understood when read in conjunction with the appended drawings. For the purpose of illustrating the invention, two embodiments of the airless spray container that are presently preferred are shown in the drawings. It should be understood, however, that the invention is not limited to the precise arrangements and instrumentalities shown.

In the drawings:

FIG. 1 is a view of a preferred airless pump bottle in accordance with one embodiment of the present invention.

FIG. 2 is a view of a second preferred airless spray container, a bag-on-valve system, in accordance with a second embodiment of the present invention.

### DETAILED DESCRIPTION OF THE PREFERRED EMBODIMENT

A preparation of an antipruritic drug for a topical application according to an embodiment of the present invention includes an aloe vera liquid base and a therapeutic amount of promethazine hydrochloride. The aloe vera liquid is a carrier for the promethazine hydrochloride. More preferably, the aloe vera liquid comprises aloe leaf juice, potassium sorbate, sodium benzoate, and citric acid to prevent bacteria growth. It is preferable for the promethazine hydrochloride to be dissolved in the aloe vera liquid.

Further to this embodiment, promethazine hydrochloride is present at a level preferably from about 0.5% to about 15% by weight, although the lower bound is flexible to the extent that the resulting preparation contains a therapeutic amount of promethazine hydrochloride. The preparation according to this embodiment preferably includes aloe vera liquid at a level from about 85% to about 99.5% by weight.

In accordance with an embodiment of the present invention, the preparation of the present invention is packaged in an airless spray container. Preferably, the airless spray container is an airless pump bottle. More preferably, the airless spray container is filled with between about 5 mL and about 325 mL of the topical promethazine hydrochloride preparation. This preferred embodiment provides enough topical promethazine hydrochloride preparation for many uses.

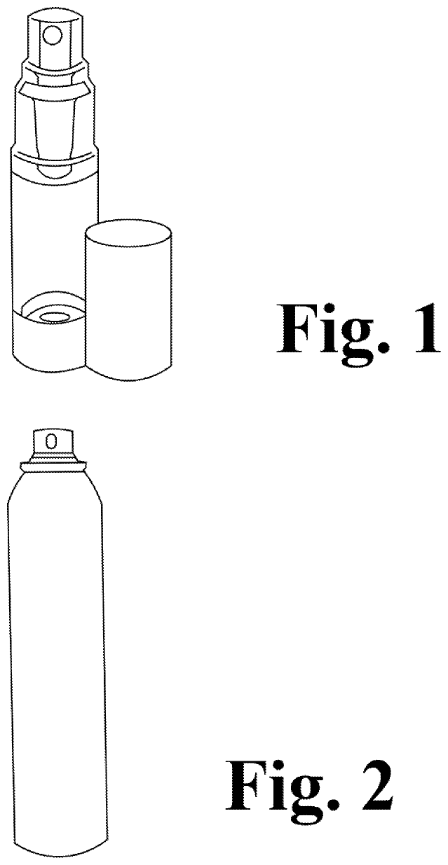

Referring now to FIGS. 1 and 2, the topical promethazine hydrochloride preparation may preferably be packaged in an airless spray container in which the topical promethazine hydrochloride preparation is injected into the package. Airless containers prevent the promethazine hydrochloride from oxidizing and turning blue, thus prolonging the shelf life of the promethazine hydrochloride preparation. It is preferable for the airless packaging to be an airless pump bottle shown in FIG. 1 that utilizes a mechanical pump in a bottle. As the pump is pushed down, the disc in the bottle rises to push the promethazine hydrochloride preparation out of the pump. The topical promethazine hydrochloride preparation may also be equally preferably packaged in a bag-on-valve system shown in FIG. 2 in which the container consists of an aerosol valve with a welded bag and the promethazine hydrochloride preparation is placed inside the bag while the propellant is filled in the space between bag and can. The promethazine hydrochloride preparation is dispensed by the propellant squeezing the bag when the spray button is pressed and the promethazine hydrochloride preparation is squeezed out of the bag by the compressed air/nitrogen, which creates the dispensing as a spray.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Example 1

### Promethazine Hydrochloride (0.75%) with Aloe Vera Liquid (99.25%)

Approximately 99.25 mL aloe vera liquid is added to a glass container. To this aloe vera liquid, 0.75 g promethazine hydrochloride is slowly added and the mixture is stirred with a mechanical stirrer until the promethazine hydrochloride has fully dissolved. The liquid is stored in airless containers. When prepared, the liquid is clear, and the liquid remains clear at ambient temperatures.

### Example 2

### Promethazine Hydrochloride (5%) with Aloe Vera Liquid (95%)

Approximately 95 mL aloe vera liquid is added to a glass container. To this aloe vera liquid, 5 g promethazine hydrochloride is slowly added and the mixture is stirred with a mechanical stirrer until the promethazine hydrochloride has fully dissolved. The liquid is stored in airless containers. When prepared, the liquid is clear, and the liquid remains clear at ambient temperatures.

### Example 3

### Promethazine Hydrochloride (10%) with Aloe Vera Liquid (90%)

Approximately 90 mL aloe vera liquid is added to a glass container. To this aloe vera liquid, 10 g promethazine hydrochloride is slowly added and the mixture is stirred with a mechanical stirrer until the promethazine hydrochloride has fully dissolved. The liquid is stored in airless containers. When prepared, the liquid is clear, and the liquid remains clear at ambient temperatures.

### Example 4

### Promethazine Hydrochloride (15%) with Aloe Vera Liquid (85%)

Approximately 85 mL aloe vera liquid is added to a glass container. To this aloe vera liquid, 15 g promethazine hydrochloride is slowly added and the mixture is stirred with a mechanical stirrer until the promethazine hydrochloride has fully dissolved. The liquid is stored in airless containers. When prepared, the liquid is clear, and the liquid remains clear at ambient temperatures.

### Conclusion, Ramification, and Scope

Thus the reader will see that an embodiment of the topical antipruritic provides a spray that is long-lasting without the need for reapplication, does not leave the skin feeling sticky or messy, is inexpensive to manufacture, and requires little packaging compared to existing products.

While my above description contains many specificities, these should not be construed as limitations on the scope, but rather as an exemplification of one embodiment thereof. Many other variations are possible. For example, water or other solvents can be used instead of aloe vera liquid as a base. Colorings, scents, and preservatives can be added. The container can be larger or smaller. The embodiment can be used to treat other causes of itching, such as yeast infections.

Accordingly, the scope should be determined not by the embodiment illustrated, but by the appended claims and their legal equivalents.

I claim:

1. A topical pharmaceutical preparation suitable for use as an antipruritic comprising an aloe vera liquid base and a therapeutic amount of promethazine hydrochloride.

2. The preparation of claim 1 wherein said promethazine hydrochloride comprises from about 0.5% to about 15% by weight of the preparation.

3. The preparation of claim 1 wherein said promethazine hydrochloride comprises from about 1% to about 10% by weight of the preparation.

4. The preparation of claim 1 wherein said promethazine hydrochloride comprises from about 1.5% to about 5% by weight of the preparation.

5. The preparation of claim 1 wherein said aloe vera liquid comprises from about 85% to about 99.5% by weight of the preparation.

6. The preparation of claim 1 wherein said aloe vera liquid comprises from about 90% to about 99% by weight of the preparation.

7. The preparation of claim 1 wherein said aloe vera liquid comprises from about 95% to about 98.5% by weight of the preparation.

8. A method of preparing a topical pharmaceutical preparation suitable for use as an antipruritic comprising the step of adding a therapeutic amount of promethazine hydrochloride to aloe vera liquid.

9. A method of preparing a topical pharmaceutical preparation suitable for use as an antipruritic comprising the steps of (i) adding a therapeutic amount of promethazine hydrochloride to aloe vera liquid; and (ii) providing said preparation as a spray.

10. The method of claim 9 wherein said providing step is accomplished through the use of an airless spray container that contains from about 0.025 g to about 1.625 g promethazine hydrochloride.

11. The method of claim 8 further comprising the step of adding purified water.
